(19) United States
(12) Patent Application Publication
(10) Pub. No.: US 2024/0307102 A1
(43) Pub. Date: Sep. 19, 2024

(54) ARCH BAR WIRE APPLICATOR

(71) Applicant: Peter Georgius, Noosa Heads, Queensland (AU)

(72) Inventor: Peter Georgius, Noosa Heads, Queensland (AU)

(21) Appl. No.: 18/578,295

(22) PCT Filed: Aug. 4, 2021
(86) PCT No.: PCT/AU2021/050851

§ 371 Date: Jan. 10, 2024

(51) Int. Cl. A61B 17/88; A61B 17/06; A61B 17/34

(52) U.S. Cl. CPC A61B 17/8861; A61B 17/3403; A61B 2017/061

(57) ABSTRACT

Disclosed is a system and method for attaching an arch bar wire to secure an arch bar. The system comprises an alignment tool onto which two needles or two needle assemblies are adapted to be fitted. The alignment tool includes a needle guide adapted to guide the position of the needles. In the assembled position, the two needles have openings which face each other, such a wire can be manipulated to enter one needle from a proximal side which is outside of the patient's mouth, exit the needle's opening from the distal side, guided to enter the opening on the distal side of the second needle, the exit the second needle on the proximal side. Thus, the exposed ends of the wire will remain on the proximal side.

### TECHNICAL FIELD

This disclosure relates to a surgical tool, in particular to applicators for arch bar wires used in the attachment of arch bars, in surgeries involving the jaw or the mandible, such as oral and maxillofacial, or mandibular surgeries.

### BACKGROUND ART

Some surgeries involving the oral cavity and the jaw, such as standard treatments of fractured jaws, involves immobilization of the jaw. The immobilization is achieved by wiring an arch bar to the jaw. Arch bars also help encourage the jaws to heal in the correct alignment. The attachment of an arch bar requires, from the front of the jaw, passing a wire through the arch bar, between the teeth and over the gum, around the back of the teeth, and then passing the wire back from behind the teeth, through a gap between the teeth, toward the front and through the arch bar again. The two ends of the wire are then twisted around in front of the arch bar securing it to the jaw.

The process of manipulating the wire, passing it between adjacent teeth from the front to back, and then back to front again, requires handling of the wire by the surgeon's fingers inside the mouth, where the wire is out of view. The process places the surgeon at risk of percutaneous injury by the wire which has been reported as high as 21%.

It is to be understood that, if any prior art is referred to herein, such reference does not constitute an admission that the prior art forms a part of the common general knowledge in the art, in Australia or any other country.

### SUMMARY

In a first aspect, the present invention provides a needle, comprising an orientation guide along a middle extent of the needle, a first needle portion extending from a first end of the orientation guide, and a second needle portion extending from a second end of the orientation guide, the first and second needle portions being hollow, wherein the orientation guide includes a locking feature, which in use is adapted to cooperate with a corresponding locking mechanism provided on an alignment tool configured to cooperate with the needle.

The first and second needle portions can be portions of a needle.

The first needle portion can be a or part of a non-coring needle.

The locking feature can be provided on a surface of the orientation guide which is angled with respect to a longitudinal axis of the needle.

The surface can be at an acute angle with respect to the longitudinal axis.

The needle can include a rotational lock which in use is adapted to cooperate with the alignment tool configured to cooperate with the needle to prevent a rotational movement of the needle with respect to the alignment tool.

The rotational lock can extend from the first end of the orientation guide, at a position which is offset from the first needle portion.

The rotational lock can be of a shorter length compared with the first needle portion.

In a second aspect, the present invention provides an alignment tool adapted to cooperate with a first needle and a second needle provided in accordance the first aspect above, comprising:
a needle positioner which is in use adapted to guide a position of the first needle in relation to the alignment tool, and to guide a position of the second needle in relation to the alignment tool, into a wire return guide located at or adjacent a first longitudinal end of the alignment tool; and
a locking mechanism which is configured to cooperate with a locking feature provided on the orientation guide of the needle, wherein the locking mechanism is movable between an engaged configuration to engage the locking feature, and a free configuration where it does not engage the locking feature.

The wire return guide can be adapted to guide a wire which is fed through the first needle into the second needle.

The wire return guide can have a wire guiding recess, wherein an opening of the recess is provided across a transverse axis which is perpendicular to a longitudinal axis of the alignment tool.

The wire return guide can have a curved surface.

The needle positioner can be located at a position which is offset from the first longitudinal end of the alignment tool.

The needle positioner can have at least two needle apertures spaced apart from each other. Three or more needle apertures can be provided.

The apertures can be provided at fixed positions in the guide.

There can be two or more apertures or sets of apertures that have adjustable positions.

The needle guide can further have locking apertures, which is in use adapted to cooperate with a rotational lock in the needle, to prevent a rotation of the needle in relation to the alignment tool.

The locking mechanism can have two separately movable parts, wherein the parts are adapted to be in the free or engaged configuration, independent of each other.

In a third aspect, the invention provides a needle holder adapted to support the needle mentioned in the first aspect above, comprising a body, the body supporting a an elongate portion, the elongate portion in use being adapted to enter a hollow needle portion of the needle.

The longitudinal axes of the elongate portion and the body may be parallel.

The body can have an opening, from which the elongate portion protrudes or extends, the opening being dimensioned to receive the hollow needle portion into an interior of the body.

The needle holder can have a sleeve which extends from the body and which is located adjacent the longitudinal extension.

The sleeve can be adapted to partially fit over an orientation guide of the needle.

The sleeve can be configured as a channel, having a channel opening which generally faces toward the longitudinal extension.

The sleeve can be provided at a non-parallel angle to the longitudinal body.

The sleeve can be adapted to partially engage the orientation guide by friction.

In a fourth aspect, the invention provides a wire manipulation tool, comprising: a clamp portion, comprising a first side surface and a second side surface which is adapted to be manipulated to close and secure a wire; a tube portion extending from the clamp portion, and being adapted to fit over a length of a needle; and a handle connected from the clamp portion so as to control the clamp portion.

The first and second side surfaces of the clamp portion can be biased closed.

The handle can comprise two handle portions that are pivotable with respect to each other, to open or close the clamp portion.

The handle portions can be pivotable about a pivot, and the clamp portion is located adjacent the pivot.

In a fifth aspect, the invention provides a needle removal tool adapted to cooperate with the needle mentioned in the first aspect above, comprising: a tube portion which is adapted to fit over at least a part of the second needle portion of the needle; a hollow body portion which is in communication with the tube portion; a lock which is movable toward or away from the hollow body portion; and a handle to manipulate the lock.

In a sixth aspect, the invention provides a method of operating the alignment tool mentioned in the second aspect above, comprising: positioning a first and a second needle, each being as mentioned in the first aspect above, wherein the positions of the first and the second needles are adapted to be locked by the locking mechanism of the alignment tool, and wherein openings of the distal needle portions of the first and second needles are positioned to face toward each other. A wire is then placed into a proximal portion of the first needle. The wire is manipulated toward the needle guide so that the wire exits the opening of the distal needle portion of the first needle, travels along a wire return guide provided by the needle guide to enter the opening of the distal needle portion of the second needle, and exits from the proximal needle portion of the second needle. In place of a wire, a mono or braded filament, or metal, synthetic, or organic fibre can be used.

The method can include the first and second needles being placed respectively through a first and a second needle aperture provided on the needle guide.

The first and second needle apertures can be selected from three or more needle apertures.

Manipulation of the wire can be performed using the wire manipulation tool of mentioned in the fourth aspect above.

In a seventh aspect, the invention provides an apparatus for attaching arch bar wire. The apparatus includes two needles mentioned in the first aspect above, each needle having a distal end and a proximal end; and an alignment tool mentioned in the second aspect above. The needles are adapted to be fitted onto the alignment tool, such that distal openings of the needles will face a longitudinal midline of the alignment tool.

The apparatus can comprise one or more needle holder provided in accordance the third aspect above, for manipulating the needles.

The apparatus can have a wire manipulation tool provided in accordance the fourth aspect above, for manipulating the wire.

The apparatus can have a needle removal tool as mentioned in the fifth aspect above, to facilitate removal of the needles.

### BRIEF DESCRIPTION OF THE DRAWINGS

Embodiments will now be described by way of example only, with reference to the accompanying drawings in which

FIG. 1 is a side elevation view of a needle;

FIG. 2 is a side elevation view of a needle alignment tool;

FIG. 3 is a front elevation view of the needle alignment tool shown in FIG. 2;

FIG. 4 is a side elevation view of the needle alignment tool shown in FIG. 2, with the needle shown in FIG. 1 being held by the needle alignment tool;

FIG. 5 is a side elevation view of a needle holder in accordance with an embodiment of the present invention;

FIG. 6 is a side elevation view of the needle holder shown in FIG. 5, holding a needle shown in FIG. 1;

FIG. 7 is a partial perspective view of a needle positioner provided in accordance with an embodiment of the present invention;

FIG. 8 is a partial perspective view of a wire return guide provided in accordance with an embodiment of the present invention;

FIG. 9 is a cross section view of a wire return guide, the cross section being taken through the wire return channel;

FIG. 10 schematically depicts a partial side elevation of a needle positioner in accordance with one embodiment;

FIG. 11 schematically depicts a partial side elevation of a needle positioner and a locking aperture component in accordance with an alternative embodiment;

FIG. 12 is a perspective view of a needle holder provided in accordance with an embodiment of the present invention;

FIG. 13 is a plan view of a wire holder provided in accordance with an embodiment of the present invention;

FIG. 14 is a plan view of a needle removal tool provided in accordance with an embodiment of the present invention;

FIG. 15 is side elevation view of needle alignment tool provided in accordance with another embodiment of the invention;

FIG. 16 is a top plan view of two needle apparatuses arranged to face each other.

### DETAILED DESCRIPTION

In the following detailed description, reference is made to accompanying drawings which form a part of the detailed description. The illustrative embodiments described in the detailed description, depicted in the drawings and defined in the claims, are not intended to be limiting. Other embodiments may be utilised and other changes may be made without departing from the spirit or scope of the subject matter presented. It will be readily understood that the aspects of the present disclosure, as generally described herein and illustrated in the drawings can be arranged, substituted, combined, separated and designed in a wide variety of different configurations, all of which are contemplated in this disclosure.

In the below, the descriptors “distal” and “proximal” are used in relation to the user's hand. However, these descriptors may be reversed, if the definition of distal is changed to mean closer to the jaw.

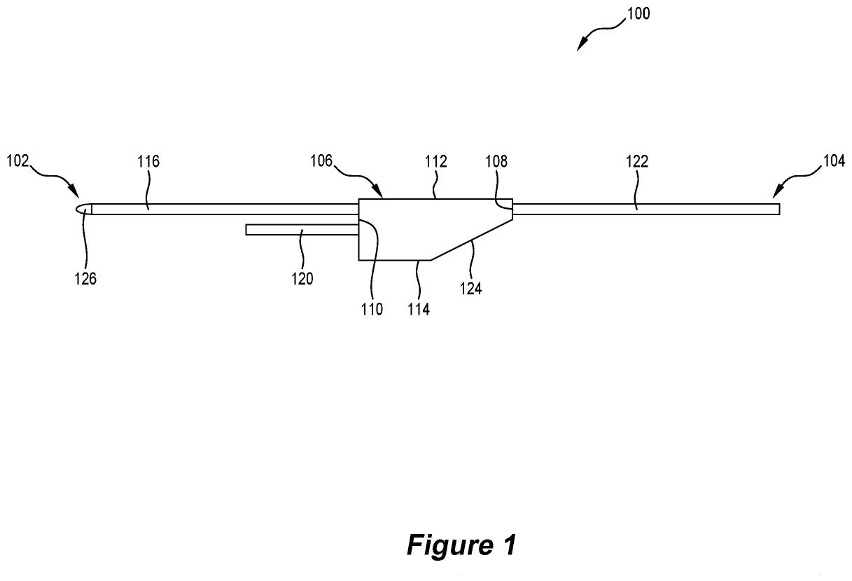

FIG. 1 depicts a needle apparatus (or needle assembly) 100 provided in accordance with as aspect of the invention. The needle apparatus 100 is part of a system provided in accordance with the invention, to thread the wire during the procedure to secure the arch bar to the jaw. The needle apparatus 100 has a distal end 102 which in use will be adapted to be located closer to the jaw, and an opposite, proximal end 104 which in use will be adapted to be located farther from the jaw.

The needle apparatus 100 includes an orientation guide 106, a hollow distal needle 116, and a hollow proximal needle 122 opposite to the distal needle 116 with the orientation guide 106 therebetween. The orientation guide 106 can be provided along a central extent of the needle apparatus 100. The orientation guide 106 is configured to have a proximal end 108 and a distal end 110.

When viewed in the side view as shown in FIG. 1, the orientation guide 106 includes, between the distal and proximal ends 108, 110, a first portion 112 and a second portion 114 which connect the distal and proximal ends 108, 110.

In the embodiment shown, the first portion 112 is located closer to the distal needle portion 116. The second portion 114 is located closer to a locking portion 120 (described below) of the needle apparatus 110. In some embodiments, the first portion 112 and the distal needle portion 116 are located on one side of a longitudinal midline of the needle apparatus 100, and the second portion 114 and the locking portion 120 are located on the other side of the longitudinal midline.

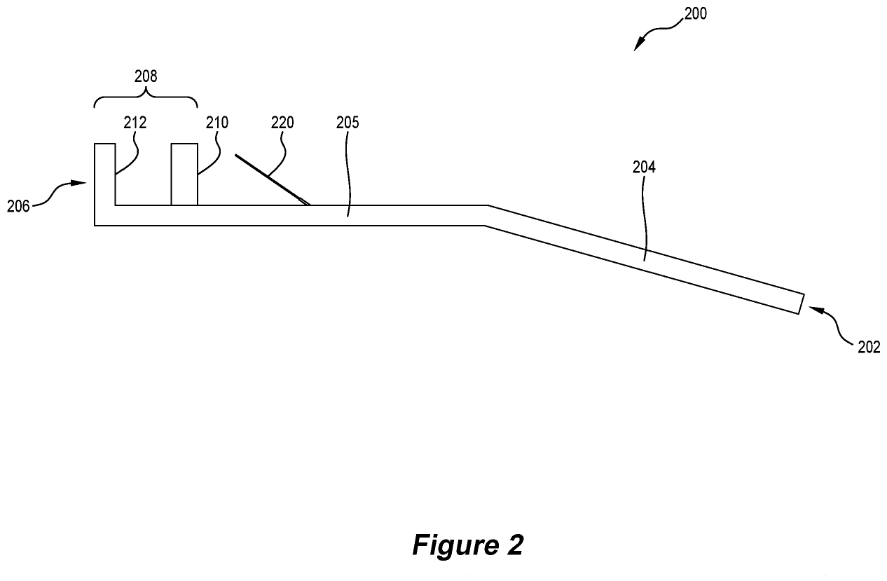

When viewed in side view (as in FIG. 1), the first portion 112 generally has a longitudinal extent, as measured in the longitudinal direction of the needle apparatus 100, which is longer than that of the second portion 114. However, this difference in the extents is not an essential requirement. At the proximal end 108 of the orientation guide 106, the two portions 112, 114 are connected by a proximal surface 124. Where the longitudinal extents of the two portions 112, 114 are different, when viewed in side view the proximal surface 124 will appear to be at a slanted angle from both portions 112, 114. As will be explained later, the proximal surface 124 is adapted to provide an engagement surface, to engage with an engagement mechanism 220 (see FIG. 2) on the needle alignment tool 200.

The distal needle portion 116 extends from the distal end 110 of the orientation guide 106 towards the distal end 102 of the assembly 100. The distal needle portion 116 is positioned so that it is aligned with a midline 118 through the orientation guide 106. There is also a locking portion 120 which extends from the distal end 110 of the orientation guide 106 toward the distal end 102 of the needle apparatus 100. The distal needle portion 116 is also positioned so to align with a midline of the orientation guide 106. The distal needle portion 116 in this embodiment is longer than the locking portion 120. As will be explained in more detail later, the locking portion 120 helps to rotationally lock the needle apparatus 100 with respect to an alignment tool 200.

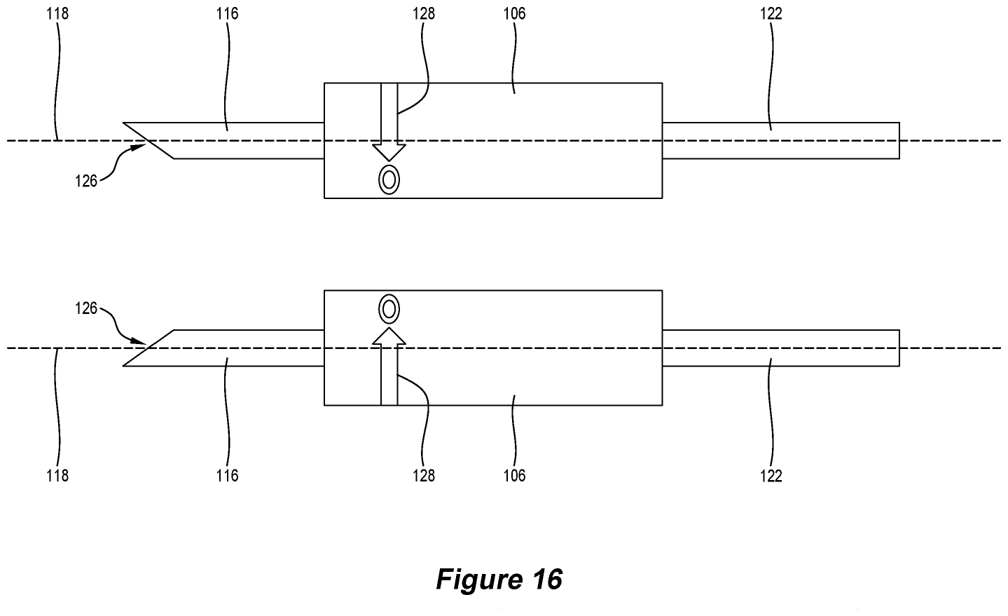

As shown in FIG. 16, the distal needle portion 116 is angularly oriented, so that its opening 126 faces (i.e., opens toward) one lateral side of the midline 118. The orientation guide 106 preferably includes a marking or a visual indication 128 to show the side of the midline which the opening 126 faces. As will be explained later, in use, two needle apparatuses 100 will be included for assembly onto a needle alignment tool 200. The two needle apparatuses 100 will include distal needle portions 116 which faces toward each other when assembled onto the needle alignment tool 200, so that their respective openings 126 face toward each other.

Referring back to FIG. 1, the proximal needle portion 122 extends from the proximal end 108 of the orientation guide 106 toward the proximal end 104 of the needle apparatus 100. The distal needle portion 116 is generally positioned opposite to the proximal needle portion 116, extending from the distal end 110 of the orientation guide 106 toward the distal end 102 of the needle apparatus 100.

The needle portions 116, 122 are coextensive, so that a wire passing into the proximal needle 122 can, or can be manipulated to, enter the distal needle 116, or vice versa. The distal and proximal 116, 122 can be separate needles. Alternatively they can be of a unitary construction, or portions of the same needle which extends through the orientation guide 106.

The distal needle 116 is preferably a non-coring needle, however a coring needle may be used.

The orientation guide 106 is configured to maintain the orientation of the distal needle 116, in relation to a needle alignment tool 200 for aligning the needle apparatus 100, so that the opening 126 of the distal needle 116 faces toward a longitudinal midline 250 of the needle alignment tool 200 (see FIG. 16). The orientation guide 106 helps prevent a rotation of the needle apparatus 100, in relation to the needle alignment tool 200, once the needle apparatus 100 is engaged with the needle alignment tool 200.

FIG. 2 depicts in side view one embodiment of the needle alignment tool 200. The needle alignment tool 200 comprises, adjacent a proximal end 202 thereof, a handle 204 by which a user can manipulate the needle alignment tool 200. The handle 204 can be angled from a main body 205. That is, a longitudinal axis of the handle 204 can be at an angle relative to an axis of the main body 205. At its distal end 206, the needle alignment tool 200 comprise a needle guide 208. The needle guide 208 is located and configured, so that in use it will guide each distal needle portion 116 to the required position, in order for the wire to be threaded in the correct position in relation to the patient's jaw, to secure the arch bar.

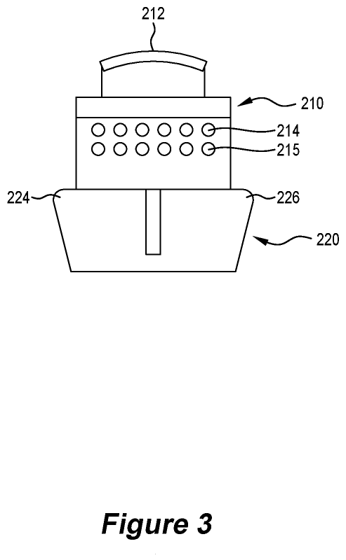

Referring to FIG. 1 and FIG. 3, the needle guide 208 includes a needle positioner 210 and a wire return guide 212. The wire return guide 212 is located at or near the distal end 206 of the needle alignment tool 200. In use, the wire return guide 212 will be positioned behind the tooth around which the arch bar wire is to pass. The needle positioner 210 is located a small distance away from the wire return guide 212, toward the proximal end 202 of the needle alignment tool 200. The needle positioner 210 in use will be positioned in front of the tooth.

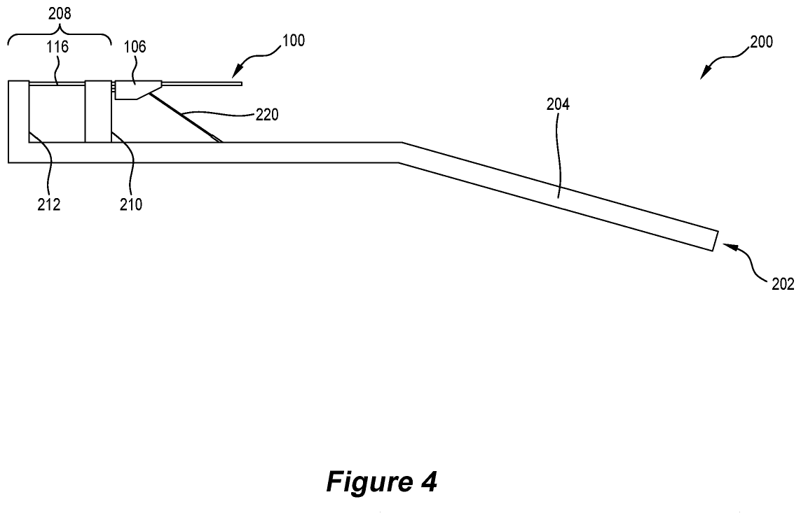

As will be discussed later, in use, the needle alignment tool 200 will be used with two needle assemblies 100—one to feed the wire from in front of the tooth to behind the tooth, and one for the wire to return from behind the tooth exiting in front of the tooth. The needle positioner 210 thus includes at least two needle apertures 214, one for the positioning of the distal needle portion 116 of each needle apparatus 100. The needle apertures 214 are positioned in a row and are each of the same height relative from the main body 205 of the needle alignment tool 200. In use, the distal needle portion 116 will be inserted through one of these apertures and advanced toward the wire return guide 212 (see FIG. 4).

In further preferred embodiments, three or more needle apertures 214 are provided, to allow adjustability in the spacing between the two distal needle portions 116. This arrangement allows a choice of the needle locations and therefore the spacing between the needles, as appropriate to match the width required for the individual target entry and exit sites of the wire either side of the selected tooth. The multiple apertures can be provided at regular intervals.

More preferably, the needle positioner 210 further includes a plurality of locking apertures 215. In the example depicted in FIG. 11 and FIG. 16, the locking apertures 215 are located closer to the handle 204, and the needle apertures 214 are located farther from the handle 204. Whereas the needle apertures 214 are intended for a distal needle portion 116 to be inserted therethrough, the locking apertures 215 are for insertion by the locking portion 120 which is provided adjacent the distal needle portion 116. Therefore, to cooperate with the embodiment of the needle apparatus shown in FIG. 1, the locking apertures 215 are located at a lower height compared with the needle apertures, when measured from the main body 205 of the needle alignment tool.

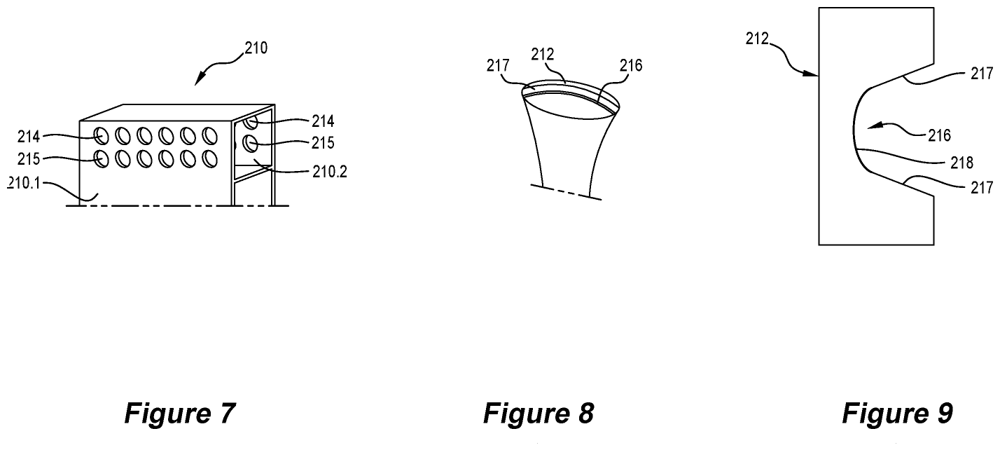

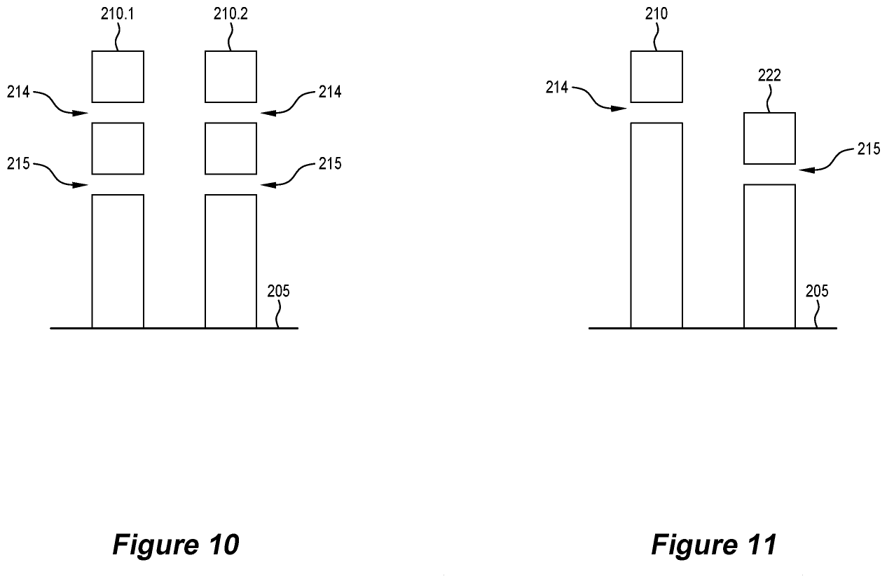

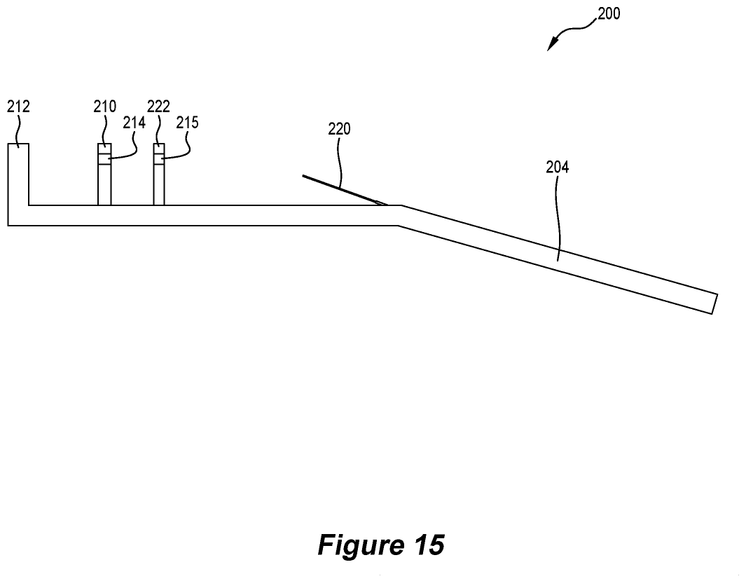

As shown in FIG. 7 and FIG. 10, in one embodiment, the needle positioner 210 includes sets of aligned needle apertures 214, and sets of aligned locking apertures 215. In this embodiment, cooperating needle apertures 214 (or locking apertures 215) which are aligned with each other are provided on a different one of two spaced part positioner portions 210.1, 210.2. This arrangement helps for the needle apparatus 100 to be stably positioned on the needle positioner 210. In the embodiment shown in FIG. 11 and FIG. 15, the needle apertures 214 are provided by openings which extend through a single body of the needle positioner 210. Similarly, the locking apertures 215 are provided by openings which extend through a separate body (a “locking aperture component 222”). In another embodiment, the needle apertures 214 and the locking apertures 215 may alternatively be provided by through openings extending through the same body or component. As can be seen in FIG. 3 and FIG. 8, and FIG. 9, the wire return guide 212 includes a guide channel or groove 216, which restricts the movement of the wire therein, while the wire is moved in relation to the wire return guide 212. The channel or groove 216 can be a recessed area which takes another form, as long as it functions to provide or define the route in which the wire is moved in the wire return guide. For simplicity, hereafter the channel or groove 216 will be referred to as the “wire route recess”. The opening of the wire route recess 216 faces toward the needle positioner 210, so as to accept the advance of the distal needle portions 116 into the wire route recess 216. Therefore, the wire route recess 216 is in alignment with the needle apertures 214 on the needle guide 208. The opening of the wire route recess 216 is preferably surrounded or defined by a chamfered edge or wall 217, where the chamfer helps to guide the distal needle portion 116 into the wire route recess 216. The end wall 218 of the wire route recess 216 will stop or limit the advance of the distal needle portion 116. The wire route recess 216 will be of sufficient width, as measured across (rather than along) the longitudinal axis of the needle alignment tool 200, to accommodate both of the distal needle portions 116 required for wire entry and for wire exit, plus the spacing between the needle portions 116. In use, the distal needle portions 116 will be provided in such an orientation that their respective openings 126 face toward each other. This allows a wire within the distal needle portion 116 of a first needle apparatus 100 to be pushed toward the wire return guide 212, moved along the wire return guide 122 within the wire route recess 216, and enter the opening 126 of the distal needle portion 116 of a second needle apparatus 100.

In some embodiments, as can be seen from FIG. 8, the wire return guide 212 has a curved shape, such as a crescent shape. The wire route recess 216 thus also has a curved or crescent shape, where the end wall 218 (see FIG. 9) of the recess is curved. This curvature helps facilitate the travel of the inserted wire along the end wall 218, from the distal needle portion 116 of one needle apparatus 100 to the distal needle portion 116 of the other needle apparatus 100.

As shown in FIG. 3, FIG. 1, and FIG. 4, the needle alignment tool 200 further includes an engagement mechanism 220. The engagement mechanism 220 is located between the handle 204 and the needle guide 208. It is spaced from the needle guide 208 in order to accommodate the orientation guide 106 on the needle apparatus.

The engagement mechanism 220 is movable with respect to the remainder of the needle alignment tool 200. It is movable between a free configuration in which it does not lock the needle apparatus 100 on the needle alignment tool 200, and an engaged configuration in which it is adapted to lock the needle apparatus 100 on the needle alignment tool 200.

As shown in FIG. 3, the engagement mechanism 220 in accordance with on embodiment includes two separately movable parts 224, 226. Each of the parts 224, 226 can be in the free or engaged configuration, independently of each other. Each part 224, 226 is adapted to cooperate with a separate needle apparatus 100.

FIG. 4 depicts the needle apparatus 100 which is held in a locked or engaged fashion by the needle alignment tool 200. The engagement mechanism 200 is of sufficient length so as to engage the orientation guide 106. It is also generally angled toward the needle guide 208, so that it can be in position to engage the orientation guide 106 on the needle apparatus 100. The engagement prevents the movement of the needle apparatus 100 toward the proximal end 202 of the needle alignment tool 200.

In the depicted embodiment, the engagement mechanism 220 engages the proximal surface 124 of the orientation guide 106. For example, the proximal surface 124 may have formed therein a recess or groove which is adapted to catch the engagement mechanism 220, or which is adapted to function as a lock which accepts the engagement mechanism 220 which will function as a key. The recess(es) and groove(s) are optional. The proximal surface 124 can instead be a flat surface which engages the engagement mechanism 220 by friction.

In use, the needle apparatus 100 is advanced toward the distal end 206 of the needle alignment tool 200, until the distal needle 116 is prevented by the end wall 218 of the wire route recess 216 from advancing further. The locking portion 120 will at this point be at least partially inserted into the locking apertures 215 provided in the needle positioner 210, thus preventing the needle apparatus 100 from making rotational movements.

The orientation guide 106 will thereby be stably positioned on the needle alignment tool 200, by the locking portion 120 (preventing rotational movement) and the engagement mechanism 220 (preventing longitudinal movement). As the needle apparatus 100 is prevented from any longitudinal movement, or rotational movement about its longitudinal axis, it is at this stage engaged or loaded onto the needle alignment tool 200.

The loading of the needle apparatus 100 onto the needle alignment tool 200 is preferably assisted by a needle holder 300. The needle apparatus 100 is adapted to be held using the needle holder 300. The operator uses the needle holder 300 to manipulate the needle apparatus 100, so as to locate the needle apparatus 100 with respect to the needle alignment tool 200.

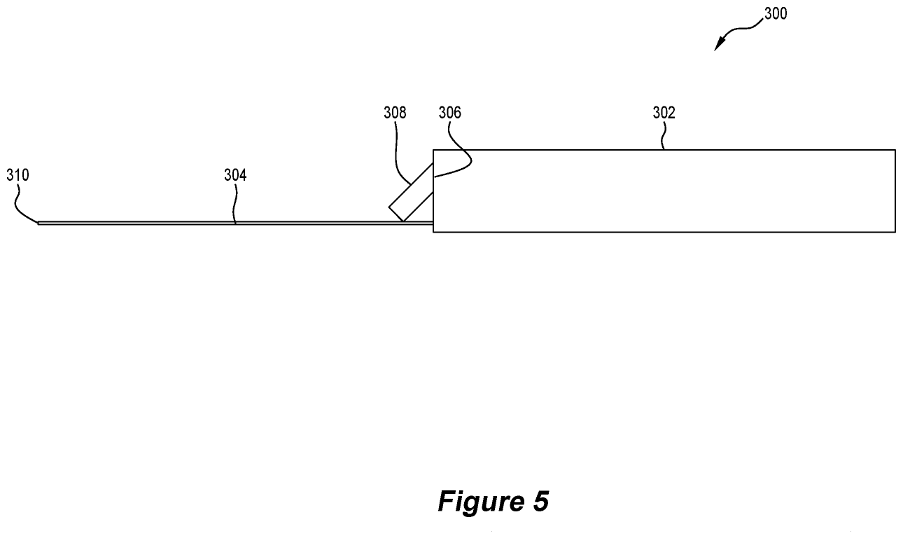

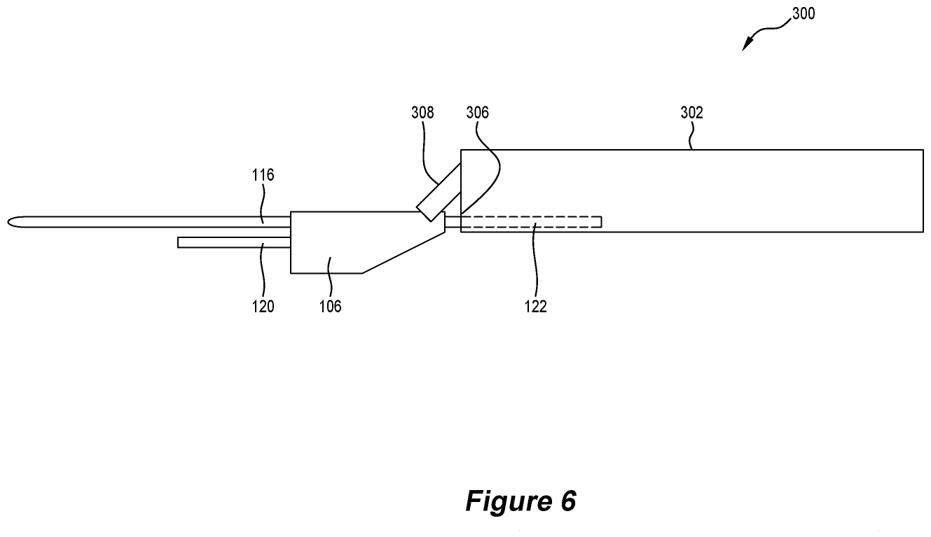

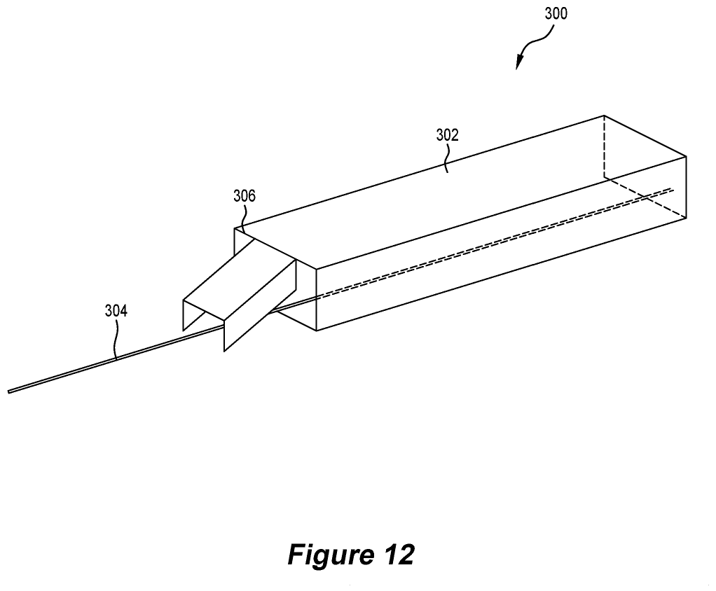

FIG. 5 and FIG. 12 depict a needle holder 300 provided in accordance with one embodiment. The needle holder 300 includes a body 302 which provides a handle that the user can manipulate. The body 302 is generally an elongate body. An elongate portion 304 such as a stylet or a probe extends or protrudes from, or is supported by, the body 302. The elongate portion 304 is dimensioned so that in use it is adapted to pass into the hollow interiors of the proximal and distal needle portions 122, 116. Continued insertion of the elongate portion 304 into the needle apparatus 100 will position the proximal needle portion 122 into the body 302 of the needle holder 300, until further advancement of the needle apparatus 100 toward the needle holder 300 is prevented by the respective dimensions of the components (see FIG. 6). The elongate portion 304 helps to reinforce the stiffness of the needle apparatus 100, for more secure handling. In this respect, the elongate portion 304 provides the function of a core that converts the hollow bore of the needle in the needle apparatus 100 into a solid needle.

In one embodiment, the needle holder 300 includes an optional sleeve 308 adjacent the distal end 306 of the body 302 of the needle holder 300. As shown in FIG. 12, the sleeve 308, in one example, is of a channel configuration. The sleeve 308 is provided at an angle from the needle holder body 302, where its “channel opening” is lengthwise and faces toward the extension 304. The channel opening in the sleeve 308 is further sized and dimensioned to receive the distal orientation guide 106, when the holder body 302 is manipulated to fit over the proximal needle portion 122. The sleeve 308 is dimensioned, to engage the orientation guide 106 by friction. This engagement also helps to hold the needle apparatus 100 in place in relation to the needle holder 300. The sleeve 308 may also be considered to function as a gusset, to reinforce the support of the needle apparatus 100 on the needle holder 300.

FIG. 6 depicts the needle apparatus 100 and the needle holder 300 where the needle apparatus 100 is loaded onto the needle holder 300. As shown FIG. 6, when loaded, the proximal needle portion 122 (shown by dashed lines) is positioned within the body 302 of the needle holder 300. To load the needle apparatus 100 onto the needle holder 300, the elongate portion 304 (obscured from view in FIG. 6) is inserted into the proximal needle portion 122, until the proximal needle portion 122 meets the distal end 306 of the needle holder body 302. Continued insertion of the elongate body 304 toward the needle apparatus 100 will cause the proximal needle 122 to enter the needle holder body 302, until this entry is impeded or prevented by the orientation guide 106 being larger than the entry not the needle holder body 302, or by an engagement of the orientation guide 106 with the sleeve 308 on the needle holder 300.

In some embodiments, a full insertion of elongate portion 304 into the needle apparatus 100 causes the tip 310 (see FIG. 5) of the elongate portion 304 to pass through the length of the distal needle portion 116, so that it is generally flush with the free tip of the distal needle portion 116. In these embodiments, the elongate extension 304 are generally of sufficient length to extend through the combined lengths of the orientation guide 106 and the distal needle portion 116.

The needle holder 300 allows for a secure handling of the needle apparatus. To manoeuvre the needle apparatus 100 by manipulating the needle holder 300, the needle apparatus 100 is first loaded onto the needle holder 300 as described above Using the needle holder 300, the needle apparatus 100 can be loaded onto the needle alignment tool 200. Thereafter, the needle holder 300 is manipulated so as to retract the elongate portion 304 out of the needle apparatus 100 and thus retract the needle holder 300 away from the needle apparatus 100. The needle apparatus 100 will engage, and therefore remain in position with respect to, the needle alignment tool 200, during this retraction. Therefore, the retraction of the needle holder 300 leaves the needle apparatus 100 engaged on the needle alignment tool 200. After the needle holder 300 is retracted, wire can be inserted into the needle apparatus.

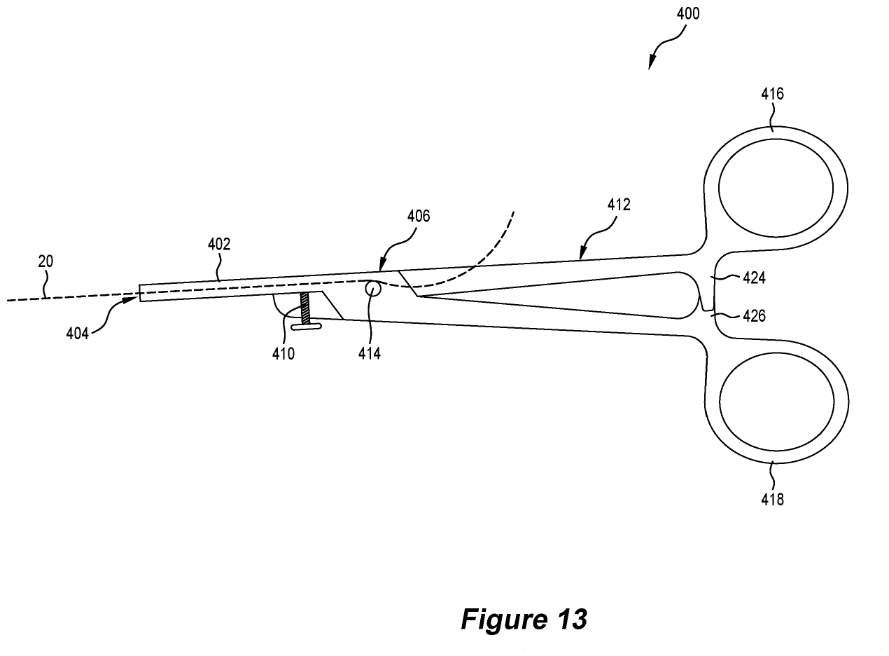

FIG. 13 depicts a wire holder 400, which can be used to facilitate the insertion of the wire 20 (shown in dashed line) into the needle apparatus 100. The wire holder 400 comprises a hollow tube 402. The hollow tube 402 has a first end opening 404, to allow the wire 20 to extend therefrom. The first end opening 404 may also be sized to pass over the proximal needle portion 122 of a needle apparatus 100. The tube 402 has a second end opening 406 generally in a longitudinally opposed position from the first end opening. From the second end opening 406, the wire 20 can be fed to the wire holder 400, and thereby into the interior of the needle 100, from the hollow tube 402. The wire 20 is adapted to be held in place, in relation to the wire holder 400, by the action of a clamping or compression device 410 to apply a compression or a clamping force. The clamping or compression device 410 may be movable toward the wire 20, to apply a compression or clamping to keep the wire 20 in place. It may be a screw or a pin, or another component shaped to perform the clamping function and dimensioned to fit within the holder 400. The clamping or compression device 410 may be a spring or spring loaded, where the spring bias keeps the wire 20 in place, and a removal of the spring bias removes the compression force. The spring-loaded version may be provided by, for example, a pin or axle which is fixed in place, but loaded with a spring which is subject to the forces applied by the user. The clamping or compression device 410 may on the hand not be spring loaded. For instance, it may include a pin, screw, rod, or the like, which is locked into a clamping position in which it applies a clamping force on the wire. The locking may be provided by e.g., a frictional engagement with a sleeve, where the pin, screw, or rod is reciprocally moved into and out of the sleeve.

The wire holder 400 further comprises a handle 412 for helping to manipulate the wire. In this embodiment, the handle 412 includes a pivot joint 414 where handle portions 416, 418 are pivotably attached. The pivot joint 414 is located adjacent the clamping or compression device 410. Forces can be applied via the pivotable handle portions 416, 418, to apply or release the clamping or compression force on the wire 20. The handle portions 416, 418 may each include a loop or ring portion, to facilitate the user to control the wire holder 400 using his or her fingers.

The handle 412 may include a mechanism to lock or delimit the position of the handle, and thus the clamping or compression device 410 which is controllable by the position of the handle 412. In this embodiment, this is done by providing each of the handle portions 416, 418 with a respective one of two cooperating part 424, 426 of an engagement or locking mechanism. The engagement may be achieved by one cooperating part 424 being retained by one or more grooves or chamfered edges in the cooperating receiving part 426. Other arrangements to achieve a locking or engagement of the handle portions 416, 416 may be used.

The parts 424, 426 are adapted to engage each other when the handles 416, 418 are in a closed position, to keep the handles 416, 418 from detaching. When the handles 416, 418 are in the closed position, the clamping or compression device 410 is correspondingly positioned or configured so that it acts upon the wire 20 to apply the compression or clamping force. An opening force sufficient to overcome the engagement mechanism 424, 426 is required to open the handles 416, 418 again to release the applied force from the wire 20. The opening force therefore will also need to apply sufficient leverage, in embodiments where the clamping or compression device 410 is a spring or is spring loaded, to overcome the spring bias.

When the wire 20 is securely held in place, the wire 20 can be positioned into the needle by advancing the needle tube 402. The handle portions 416, 418 can then be used to release the hold on the wire 20, and manipulated to move the next section of the wire into the needle. The compressive forces provided onto the wire 20 provide stiffness, so that the wire 20 can be passed forward through the needle, moved along the wire return, into the opposite needle and back out.

The wire holder 400 as described above may be purpose built, or it may be modified from existing instrumentation, such as a haemostat.

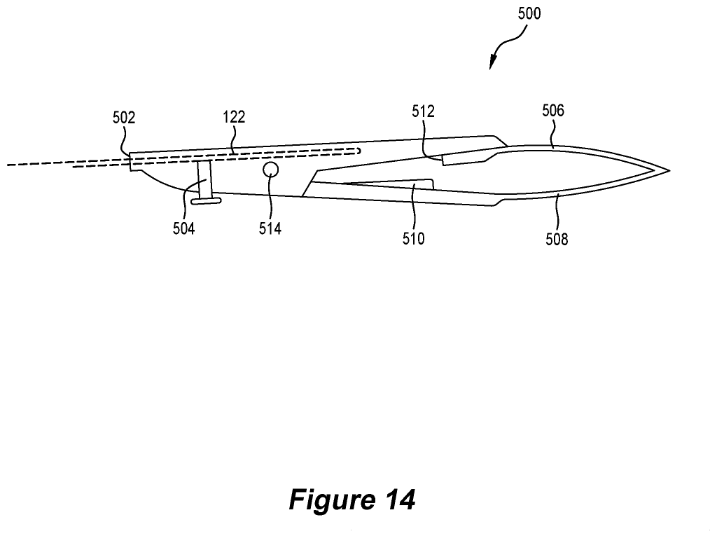

FIG. 14 depicts a needle removal tool 500. The needle removal tool 500 is used for removing the needle apparatus 100 from the needle alignment tool 200. The needle removal tool 500 includes a tube or similar part 502 having a cavity therein, dimensioned for fitting over the proximal needle portion 122 in the needle apparatus 100. The tube 502 extends from or is attached to a first handle part 506. The needle removal tool 500 includes a second handle part 508. The first and second handle parts 506, 508 are pivotable about a pivot join 514. When one or both of the handle parts 506, 508 are moved towards each other, so that the handle parts 506, 508 are closed toward each other, the needle which is inserted into the needle removal tool 500 will be secured by a clamping force. A compression part 504 is located adjacent the pivot join 514, on the other side the pivot 514 from the handle parts 506, 508. This positioning is so that forces applied by manipulating the handle parts will apply a leverage to control the compression device 504. The compression device 504 may be but is not limited to a compression screw, pin, rod, spring, or a spring-loaded device.

When the handle parts 506, 508 are in a first, “open” position, the compression device 504 is configured and positioned so as to apply a compression or clamping force on the proximal needle portion 122. A movement of the handle parts 506, 508 toward each other about the pivot 514 will cause the compression device 504 to release the force on the proximal needle portion 122. In the depicted embodiment, the handle parts 506, 508 each include or carry a cooperating portion (respectively 512, 510), which are adapted to engage or clamp onto each other, such by friction, compression, or by a cooperating arrangement, when the handle parts are in the second, “closed” position. In some embodiments, where the compression device 504 is a screw or spring or spring loaded, etc, the engagement force which keeps the handle parts 506, 508 in the “closed” position will need to apply enough leverage to overcome the bias or the device 504.

In operation, whilst the needle removal tool 500 is securely positioned onto the proximal needle portion 122, which is loaded onto the needle alignment tool 200, the engagement mechanism 220 of the needle alignment tool 200 (see FIGS. 3 and 4) can be disengaged. This can be done by, e.g. manually pushing the locking mechanism 220 into the disengaged configuration. The needle apparatus 100 is then free to be removed from the needle alignment tool 200 by moving the needle removal tool 500 away from the needle alignment tool 200.

During the surgical procedure, the needle alignment tool 200 is positioned inside the patient's mouth, so that the wire return guide 212 on the distal end of the needle alignment tool 200 is place inside the mouth, behind the tooth requiring passage of a wire. The needle positioner 210 is positioned in front of the teeth and outside of the mouth. A first needle apparatus 100 is passed through the needle positioner 210, at a chosen location 214 to match the target entry point for the wire, from the outside of the mouth. The first needle apparatus 100 is then passed through the needle positioner 210, passing between the teeth and in some cases through the gum tissues, until the distal needle portion 116 meet the wire return guide 202. Assuming the parts are properly aligned, the wire will meet the floor of the wire route recess 216. Preferably, the needle holder 300 is used to manipulate the needle apparatus 100.

The needle apparatus 100 orientation will stay in the same longitudinal and rotational position, in relation to the needle alignment tool 200. This positional stability is due to the action of the engagement mechanism 220, and where available, the engagement between the anti-rotation locking portion 120 on the needle apparatus 100 and the locking aperture 215.

The needle apparatus 100 will be oriented so that the opening 126 of the distal needle portion 116 now faces towards the midline of the needle alignment tool 200. Marking can be provided on the needle alignment tool, the needle apparatus, or both, to indicate the orientation of this opening 126 in relation to the midline of the needle alignment tool 200.

The aforementioned procedure is now repeated with the using a second needle apparatus 100, with the position of the second needle apparatus 100 in relation to the needle positioner 210 chosen to match the exit site of the wire between the teeth. The second needle apparatus 100 will be identical to the first needle apparatus 100, except that the opening 126 of the distal needle portion 116 will face in the opposite direction. In use, both needle apparatuses will thus have their openings face the midline of the alignment tool, and thus each other.

Once the needle apparatuses 100 are placed and locked in, the needle holder(s) 300 for the needle apparatuses 100 (if used to manipulate the needle apparatuses) is or are removed, leaving the needle apparatuses 100 in place on the needle alignment tool 200. A wire 20 is then pushed into the needle apparatus 100 intended for the entry of the wire, to that it can return along the wire return 212, and back towards the outside of the mouth, via the proximal needle portion 122 of the second needle apparatus for the return or exit of the wire.

The pushing of the wire 20 may be facilitated using the wire holder 400. To do so, a wire holder 400, in which the wire tube 402 is or is to be loaded with wire 20, is placed over the proximal needle portion 122 of one needle apparatus. The compression on the wire 20 is released, so that the wire can be pushed forward, and wire hold location where the compression device will hold the wire, is moved to the next section in the wire. The compression is again placed on the wire. This is repeated as the wire moves forward, eventually out of the opening 126 in the distal needle portion 116, and into the wire return recess 216. It will be understood that and the wire holder 400 can also be moved forward or backward, to adjust the position of the wire 20 with respect to the needle. The wire 20 is continued to be pushed along the wire return recess 216 and will enter the opening 126 of the second needle apparatus. The wire 20 will be pushed to enter the distal needle portion 116 of the second needle apparatus, eventually exiting the second needle apparatus 100 at the proximal end. This is repeated until the desired length of wire has exited the second needle apparatus 100.

The needle apparatuses 100 are then removed. This can be facilitated using the needle removing tool 500 and by manually disengaging the locking mechanism 220. Once both of the needle assemblies 100 have been removed, the needle alignment tool 200 can be removed. The removal of the needle alignment tool 200 leaves the wire 20 in situ. The wire 20 can then be used to attach the arch bar in the standard manner.

The surgical device described above, in each of its various embodiments, aids in the passage of the wire in the attachment of an arch bar to the jaw. The device helps to minimise the risk of percutaneous injury from the arch bar wire, as by using the device, the surgeon's fingers remain outside of the patient's mouth. This ensures that any sharp part of the wire remains within the surgeon's sight during handling. The provision of the surgical device also provides additional choices for surgeons, who may have preferences due to different suturing methods or styles, hand size, or other factors.

Variations and modifications may be made to the parts previously described without departing from the spirit or ambit of the disclosure.

For example, in the above, two needle apparatuses 100 are said to be used to provide the two distal needles that will enter the mouth-one to feed the wire to the back of the tooth, and one to accept wire returning from the back of the tooth. However, the distal needles 116 may be provided by one needle apparatus 100. In this case, only one distal needle 116 needs to be in alignment with the proximal needle 122 in the needle apparatus 100, to accept the wire being fed by the operator. The other distal needle 116 will be spaced from the first distal needle 116. The spacing may be fixed or adjustable.

As a further example, in the above, the locking apertures 215 used for preventing a rotation of the needle apparatus 100 with respect to the needle alignment tool 200 are provided on the needle positioner. In the alternative embodiment shown in FIG. 15, the locking apertures 215 are provided on a separate needle locking component 222 provided on the needle alignment tool 200. It will be appreciated that the dimensions of the needle apparatus will be correspondingly configured, so as to cooperate with the needle alignment tool 200.

Taken together, above disclosed is a system and method for attaching an arch bar wire to secure an arch bar. The system comprises an alignment tool onto which two needles or two needle assemblies are adapted to be fitted. The alignment tool includes a needle guide adapted to guide the position of the needles. In the assembled position, the two needles have openings which face each other, such a wire can be manipulated to enter one needle from a proximal side which is outside of the patient's mouth, exit the needle's opening from the distal side, guided to enter the opening on the distal side of the second needle, the exit the second needle on the proximal side. Thus, the exposed ends of the wire will remain on the proximal side.

The system may include the needle holder described, which is configured to facilitate the manipulation of the needle with respect to the needle alignment tool. A wire manipulation tool may also be included in the system, to facilitate the manipulation of the wire. The system may further include a needle removal tool as described above, to facilitate the removal of the needle from the alignment tool.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1-41. (canceled)

42. A needle apparatus, comprising:
an orientation guide along a middle extent of the needle apparatus;
a first needle portion extending from a first end of the orientation guide and a second needle portion extending from a second end of the orientation guide, one of the first and second ends being a proximal end of the orientation guide and the other one of the first and second ends being a distal end of the orientation guide, wherein in use the distal end is positioned closer to a patient than the proximal end;
the first and second needle portions being hollow;
wherein the orientation guide includes a locking feature, which in use is adapted to cooperate with a corresponding locking mechanism provided on an alignment tool configured to cooperate with the needle.

43. The needle apparatus of claim 42, wherein the first and second needle portions are portions of a needle.

44. The needle apparatus of claim 42 wherein the first needle portion is a or part of a non-coring needle.

45. The needle apparatus of claim 42, wherein the locking feature is an engagement surface provided on the orientation guide, the engagement surface being angled with respect to a longitudinal axis of the needle.

46. The needle apparatus of claim 45, wherein the surface is at an acute angle with respect to the longitudinal axis.

47. The needle apparatus of claim 42, further comprising a rotational lock which in use is adapted to cooperate with the alignment tool configured to cooperate with the needle to prevent a rotational movement of the needle with respect to the alignment tool.

48. The needle apparatus of claim 47, wherein the rotational lock extends from the first end of the orientation guide, at a position which is offset from the first needle portion.

49. The needle apparatus of claim 48, wherein the rotational lock is of a shorter length compared with the first needle portion.

50. An alignment tool adapted to cooperate with a first needle apparatus and a second needle apparatus,
each needle apparatus comprising:
an orientation guide along a middle extent of the needle apparatus, the orientation guide including a locking feature;
a first needle portion extending from a first end of the orientation guide and a second needle portion extending from a second end of the orientation guide, one of the first and second ends being a proximal end of the orientation guide and the other one of the first and second ends being a distal end of the orientation guide, wherein in use the distal end is positioned closer to a patient than the proximal end;
the first and second needle portions being hollow;
the alignment tool having a first longitudinal end and a second longitudinal end, the alignment tool comprising:
a wire return guide located at or adjacent the first longitudinal end of the alignment tool;
a needle positioner which is, in use, adapted to guide a position of the first needle apparatus in relation to the alignment tool, and to guide a position of the second needle apparatus in relation to the alignment tool, into the wire return guide; and
a locking mechanism which is configured to, in use, cooperate with the locking feature provided on the orientation guide of the first or second needle apparatus, wherein the locking mechanism is movable between an engaged configuration to engage the locking feature, and a free configuration where it does not engage the locking feature.

51. The alignment tool of claim 50, wherein each of the first and second needle apparatus comprises a respective rotational lock, and the needle positioner is configured to receive the first needle portion and rotational lock of the first needle apparatus and/or first needle portion and rotational lock of the second needle apparatus

52. The alignment tool of claim 50, wherein the wire return guide is adapted to guide a wire which is fed through the first needle apparatus, into the second needle apparatus.

53. The alignment tool of claim 50, wherein the wire return guide comprises a wire routing recess, wherein an opening of the recess is provided across a transverse axis which is perpendicular to a longitudinal axis of the alignment tool.

54. The alignment tool of claim 50, wherein the needle positioner is located at a position which is offset from the first longitudinal end of the alignment tool.

55. The alignment tool of claim 50, wherein the needle positioner includes at least two needle apertures spaced apart from each other, for in use, receiving the first needle portions of the first and/or second needle apparatus.

56. The alignment tool of claim 50, wherein the needle positioner comprises locking apertures, which is in use adapted to cooperate with a rotational lock in the needle, to prevent a rotation of the needle in relation to the alignment tool.

57. The alignment tool of claim 56, wherein the locking apertures are provided at a location which is longitudinally offset from the needle positioner.

58. A needle holder adapted to support a needle apparatus, the needle apparatus comprising:
an orientation guide along a middle extent of the needle apparatus;
a first needle portion extending from a first end of the orientation guide and a second needle portion extending from a second end of the orientation guide, one of the first and second ends being a proximal end of the orientation guide and the other one of the first and second ends being a distal end of the orientation guide, wherein in use the distal end is positioned closer to a patient than the proximal end;
the first and second needle portions being hollow;
wherein the orientation guide includes a locking feature, which in use is adapted to cooperate with a corresponding locking mechanism provided on an alignment tool configured to cooperate with the needle;
the needle holder comprising:
a body;
a longitudinal extension supported by the body, the longitudinal extension in use being adapted to enter the second needle portion of the needle apparatus.

59. The needle holder of claim 58, including a sleeve which extends from the body and which is located adjacent the longitudinal extension, adapted to at least partially fit over the orientation guide of the needle apparatus.

60. The needle holder of claim 59, wherein the sleeve is provided at a non-parallel angle to the longitudinal extension.

61. The needle holder of claim 59, wherein the sleeve is adapted to partially engage the orientation guide by friction.
